(19) United States
(12) Patent Application Publication
(10) Pub. No.: US 2024/0092637 A1
(43) Pub. Date: Mar. 21, 2024

(54) PROCESSING OF FEED STREAM USING REFRACTORY FOR HYDROGEN PRODUCTION AND REDUCED CARBON EMISSIONS

(71) Applicant: H QUEST VANGUARD, INC., Pittsburgh, PA (US)

(72) Inventors: George L. Skoptsov, Pittsburgh, PA (US); Aayush Mantri, Pittsburgh, PA (US); Vignesh Viswanathan, Pittsburgh, PA (US)

(21) Appl. No.: 18/468,302

(22) Filed: Sep. 15, 2023

Related U.S. Application Data
(60) Provisional application No. 63/375,976, filed on Sep. 16, 2022.

(51) Int. Cl. C01B 3/26; B01J 19/08; C01B 32/05; C07C 2/82

(52) U.S. Cl. CPC C01B 3/26; B01J 19/088; C01B 32/05; C07C 2/82; B01J 2219/0869; B01J 2219/0871; B01J 2219/0898; C01B 2203/1041; C01B 2203/1235

(57) ABSTRACT

Systems and methods for producing hydrogen and solid carbon from gaseous feedstock. The system includes a plasma reactor configured to receive and convert feedstock comprising hydrocarbons into acetylene-containing feed stream, and a refractory coupled to the plasma reactor that is configured to receive and decompose acetylene to hydrogen and solid carbon. The system is further configured to deliver one or more auxiliary feedstock comprising hydrocarbons directly to the refractory for decomposition into hydrogen and carbon. The energy required to decompose the auxiliary feedstock is provided by the energy released from decomposition of acetylene in the refractory.

### RELATED APPLICATIONS AND CLAIM OF PRIORITY

This patent document claims priority to U.S. provisional patent application No. 63/375,976, filed Sep. 16, 2022, the disclosure of which is fully incorporated into this document by reference.

### TECHNICAL FIELD

This disclosure is related to processing of a feed stream containing hydrocarbons and the production of hydrogen and solid carbon products.

### BACKGROUND

The use of fossil energy has fueled unprecedented economic growth and development worldwide, along with an associated increase in concentration of atmospheric CO₂. One approach to reducing CO₂ emissions is to deploy carbon capture and storage (CCS) technology with the existing fossil power plants. However, the immediate, direct costs associated with the deployment of CCS technology, primarily due to the high capital costs and energy requirements of gas separation, have been a barrier to wide-scale deployment of the technology. Incentives for industry to deploy CCS are very limited or non-existent.

An alternative to CCS is carbon capture and reutilization (CCR), where CO₂ serves as a valuable resource rather than a waste product. CO₂ is a plentiful potential feedstock for many products, including commercial chemicals, plastics, and improved cement. However, its direct applications are limited, as CO₂ is a stable compound with a low energy state and does not readily participate in chemical reactions without added energy. Additionally, the supply of CO₂ that could be available as the USA moves toward a carbon-constrained economy far exceeds the current demand for CO₂ as a commodity chemical.

Routine or continuous flaring occurs when there are no opportunities to utilize gas streams; this wastes valuable energy resources and releases carbon dioxide (CO₂) into the atmosphere. Flares are used extensively to dispose of purged and wasted products from refineries, unrecoverable gases emerging from oil wells, vented gases from blast furnaces, unused gases from coke ovens, and gaseous wastes from chemical industries. While gases flared from refineries, petroleum production, chemical industries, and coke ovens, are composed largely of low molecular weight hydrocarbons with high heating value, blast furnace and carbon black plant flared gases are largely inert species with CO or hydrogen and have low heating value, requiring combustion of additional fuel gas to maintain the flame.

Flaring combusts and oxidizes methane-rich waste gas to CO₂ and water. Although flaring waste gas is better than venting it (the global warming potential of CO₂ is approximately 84 times less than that of methane over a twenty-year period), flaring is a harmful and wasteful practice. Worldwide, flaring contributes 300 million tons of CO₂ annually, accounting for 42% of GHG emissions associated with oil and gas production. According to International Energy Agency (IEA) data, 150 billion cubic meters of natural gas were flared globally in 2019. Increased scrutiny of incomplete flare combustion and venting is also warranted: recent studies suggest extensive flaring is not only a primary source of CO₂ emissions, but also a significant source of methane (CH₄) emissions due to malfunctioning and unlit flares.

The need to reduce global greenhouse emissions further motivates adoption of renewable (e.g., solar, wind) electric power generation as well as the rapid growth in market share of electric vehicles. Both intermittent, distributed power generation and electric vehicles drive the need for high-capacity electric energy storage solutions: batteries. All batteries include carbon as the key component; lithium-ion batteries in particular require roughly 1-1.2 kg of carbon per 1 kWh of capacity; 92% of which is graphite, and 8% conductive carbon black. Despite the higher (up to 10×) cost, synthetic graphite is the preferred material owing to its higher purity and consistency.

Conventional production of precursors to synthetic graphite and of conductive carbon blacks from heavy fossil sources is challenged with high pollutant and GHG emissions (as much as 4 kg CO₂/kgC). This puts the objectives of reduced carbon emissions and growth in the use of electric vehicles and renewable energy in direct conflict.

In the United States, natural gas is used almost exclusively as feedstock for on-purpose hydrogen production in Steam Methane Reforming (SMR) units by refineries, industrial gas producers, and other chemical manufacturers. Conventional 100,000 Nm³/day SMR plant generates ˜9 kg CO₂ for each kg of hydrogen produced and consumes 350 thousand gallons of fresh water per day.

Reforming or gasification of fossil hydrocarbons is the primary (95% share) method of hydrogen production at industrial scale, which reports indicate produce 830 million tons of CO₂/year. There are two main technological approaches to low or near-zero CO₂ production of hydrogen:
1. Coupling the reforming hydrogen plants with CO₂ capture and storage systems.
2. Alternatives to reforming/water-gas-shift: electrolysis of water powered by non-carbon energy sources and direct decomposition of hydrocarbons to hydrogen and carbon. While only about a third of the natural gas in SMR plants is directly combusted for heat value, virtually all of the carbon in the feedstock is converted and emitted as CO₂.

Reduction or elimination of CO₂ from SMR necessarily requires CO₂ capture and subsequent sequestration or utilization. There is a need for a cleaner alternative to SMR.

### SUMMARY

A method for producing hydrogen is disclosed. The method includes: delivering a first feed stream into at least one plasma reactor; activating microwave pyrolysis in the plasma reactor to convert the first feed stream to an acetylene-containing stream; delivering the acetylene-containing feed stream to a refractory; activating decomposition of the acetylene-containing feed stream into hydrogen in the refractory. In various embodiments, the first feed stream may comprise one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof. In various embodiments, the first feed stream may further comprise methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

In various embodiments, the method comprises delivering a second feed stream to the refractory and decomposing the second feed stream to produce hydrogen and solid carbon in the refractory. In various embodiments, the second feed stream may comprise one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof. In various embodiments, the second feed stream may further comprise methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

In various embodiments, the temperature within the refractory may be about 800° C. to about 1600° C., or about 1000° C. to about 1300° C. In various embodiments, the refractory may include a catalyst such as a solid carbon, e.g., carbon black. In various embodiments, decomposition of acetylene-containing feed stream may produce hydrogen and solid carbon. In other embodiments, decomposition of the second feed stream may produce hydrogen and solid carbon.

Also disclosed is a system for producing hydrogen from a feed stream containing hydrocarbon the system comprising: a plasma reactor for converting the feed stream to an acetylene-containing feed stream; and a refractory coupled to the plasma reactor. In various embodiments, the feed stream may comprise one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof. In other embodiments, the feed stream may further comprise methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

In various embodiments, the system may comprise a one or more plasma reactors coupled to the refractory. In various embodiments, the refractory may comprise one inlet coupled to each plasma reactor for receiving the acetylene-containing feed stream, the inlet being at one end of the refractory, and at least one outlet at an opposite end of the refractory for expelling the hydrogen. In various embodiments, the refractory may be configured to decompose acetylene-containing feed stream to hydrogen and solid carbon. In various embodiments, the refractory may comprise one or more auxiliary inlet gas feeds configured to feed a second feed stream to the refractory. In certain embodiments, the refractory may comprise one or more inlet gas feeds configured to a second feed stream into the refractory.

In various embodiments, the second feed stream may comprise one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof. In other embodiments, the second feed stream may further comprise methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof. In various embodiments, the refractory may be configured to decompose the second feed stream to hydrogen and solid carbon.

In various embodiments, the system may further comprise a heating element attached to an outer surface of the refractory. In other embodiments, an insulating layer may enclose an outer surface of the refractory. In various embodiments, the refractory may be a conical shape, a rectangular shape, a square shape, a cylindrical shape, a converging-diverging cone shape, or a combination thereof.

### BRIEF DESCRIPTION OF THE FIGURES

FIG. 1 is a graph showing acetylene decomposition rates versus reaction time in a refractory.

FIG. 2 is a graph showing methane decomposition rates versus residence time in a refractory.

FIG. 3 is a process flow diagram for a natural gas stream passing through a plasma reactor and a refractory in a hydrogen processing plant.

FIG. 4 illustrates a non-limiting example configuration of a plasma reactor and refractory.

FIGS. 5A and 5B illustrate non-limiting example configurations of refractory.

FIGS. 6A and 6B illustrate non-limiting example configuration of a scale up of 15 plasma reactors in a 2-ring configuration and 24 plasma reactors in a 3-ring configuration coupled to conical refractory.

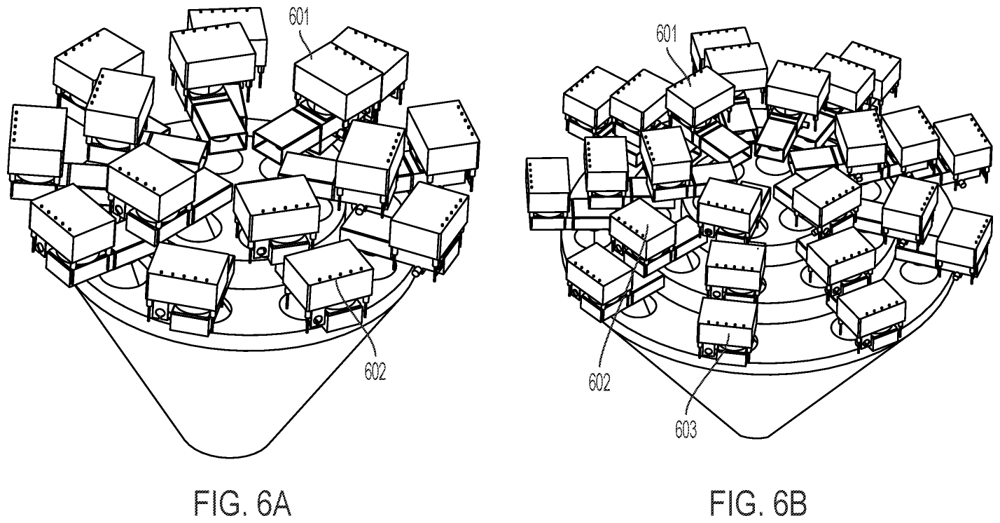

FIGS. 7A and 7B illustrate a non-limiting example configuration of a side view and top-down view of a refractory.

FIG. 8 is graph showing example outer wall temperature of a refractory alumina tube in operation.

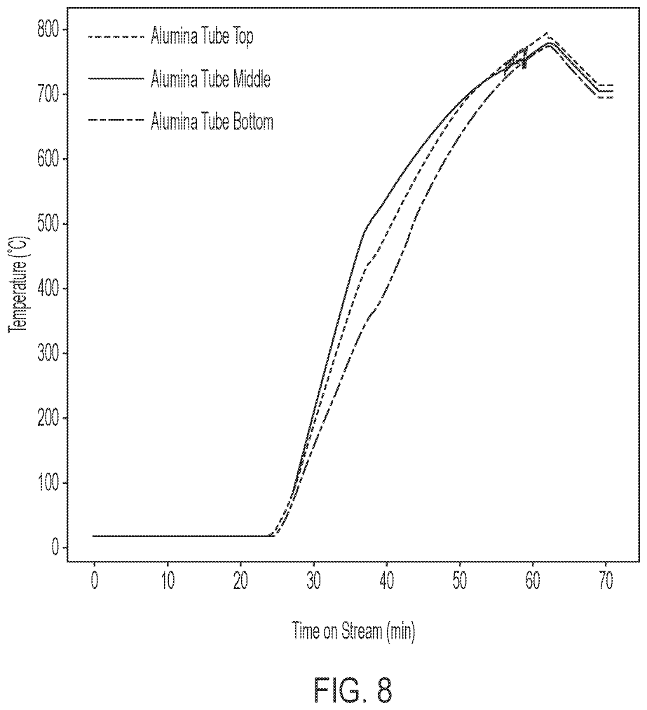

FIG. 9 shows carbon formation pathways in microwave plasma and methane pyrolysis.

FIG. 10 is a graph showing carbon selectivity vs. acetylene selectivity.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” (or “comprises”) means “including (or includes), but not limited to.” When used in this document, the term “exemplary” is intended to mean “by way of example” and is not intended to indicate that a particular exemplary item is preferred or required.

In this document, when terms such as “first” and “second” are used to modify a noun, such use is simply intended to distinguish one item from another, and is not intended to require a sequential order unless specifically stated. The terms “approximately” and “about” when used in connection with a numeric value, are intended to include values that are close to, but not exactly, the number. For example, in some embodiments, the terms “approximately” and “about” include values that are within +/−5 percent of the value, and in some embodiments values that are within +/−10 percent of the value.

In this document, the term “coupled” or “connected”, when referring to two physical structures, means that the two physical structures touch each other. Devices that are connected or coupled may be secured to each other, or they may simply touch or be operably connected each other and not be directly secured to each other.

An efficient, clean, and cost-effective process of hydrocarbon decomposition such as methane decomposition, and system for the same, are provided herein through application of focused plasma technology to produce chemically-active acetylene, which, under bulk temperatures, produces hydrogen and valuable carbon products.

Methane pyrolysis can be viewed as a single step reaction yielding hydrogen and solid carbon: CH₄→2H₂+C(s) ΔH=75.6 kJ/molCH₄

Processing 1.0 m³ of methane may yield 1.5 m³ of hydrogen and 0.5 m³ of acetylene.

However, the actual reaction kinetics are more complex. A more accurate (though still simplified) view is that of a two-stage process. First, methane loses hydrogens, yielding methyl radicals, which quickly combine to ethane and dehydrogenate to acetylene. Then acetylene, unless promptly quenched, exothermically polymerizes in a reaction known as the hydrogen abstraction, condensation of acetylene (HACA) process, releasing hydrogen and building up a mixture of polyaromatic molecules, which in limit give rise to graphene and carbon black.

2CH₄→3H₂+C₂H₂ ΔH=376.08 kJ/mol C₂H₂

C₂H₂→H₂+2C(s) ΔH=−226.88 kJ/mol C₂H₂

Methane pyrolysis requires significant energy inputs, owing to the high enthalpy of formation of acetylene, with theoretical energy requirement of about 3.26 kWh/kg CH₄. However, full decomposition of acetylene releases about 60% of its energy back into the system (226.88 kJ/mol C₂H₂), which then can drive further pyrolytic reactions of methane and sustain the high temperatures, bringing the theoretical energy requirement of complete methane decomposition down to about 1.3 kWh/kg CH₄.

In the plasma reactor (first stage of the process), methane is converted into acetylene. opening a path to decarbonization of the chemical production. This work builds on acetylene's reactivity to maximize methane conversion. Acetylene is extremely reactive and will react further to yield more hydrogen and high-structure, high-purity carbon, unless it is cooled. When the refractory is kept at a high temperature and the acetylene is maintained at a high temperature, in the second stage of the process, acetylene molecules combine and fuse to make more complex carbon structures and hydrogen is released. In this second stage, complete and highly efficient conversion to hydrogen and solid carbon and/or a mixture of polyaromatic molecules is possible.

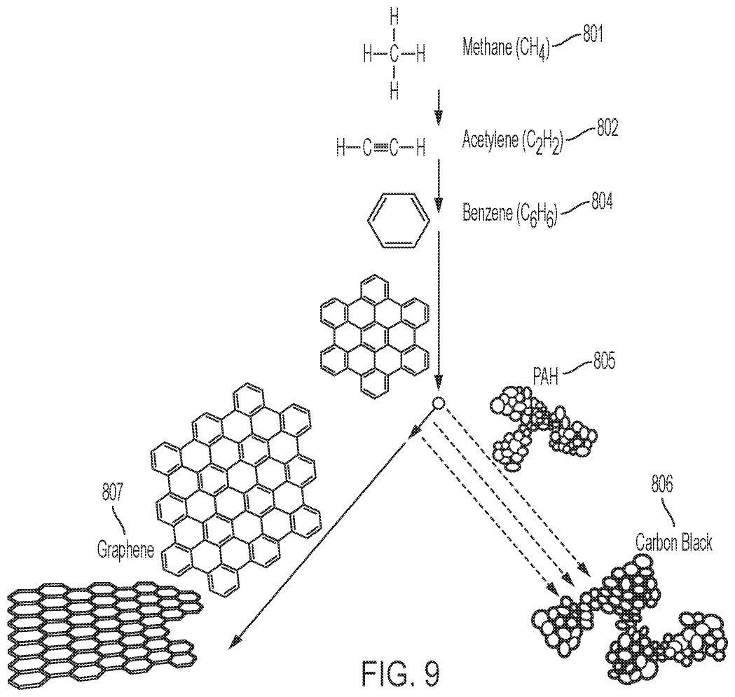

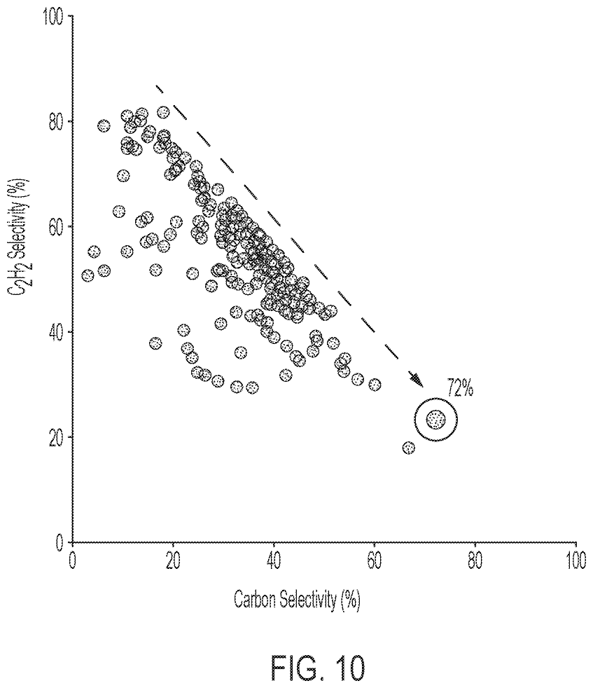

FIG. 9 shows carbon formation pathways in microwave pyrolysis. Methane 801 pyrolysis yields acetylene 804. In turn, if sufficient temperature conditions are maintained (e.g., above 800° C.), polymerizes to benzene 804, which further undergoes exothermic chain polymerization yielding either graphene sheets 811, polyaromatic hydrocarbons (PAH) 812, and/or carbon black 813 depending on operating conditions. Thermally controlled second stage maximizes the conversion rate and carbon selectivity while reducing energy requirements. As shown in FIG. 10, carbon selectivity is maximized when acetylene conversion is minimized with highest carbon selectivity of 72%.

The process of the disclosure is designed to be compatible with conventional SMR plants and is suitable for retrofitting or augmenting existing installations. The overall process produces a hydrogen stream with about 80% purity suitable for separation with conventional SMR PSA units. A small amount (1-2% feed) of process gas is needed to serve as sheath gas to prevent surface coke formation as well as to provide free electrons that are accelerated by (couple to) the microwave electromagnetic field and serve as the energy carriers. While an inert gas such as argon would be typically used in this scenario, hydrogen may also be used. Argon is expensive and imposes logistical challenges. Argon may be replaced with nitrogen and self-generated hydrogen. This eliminates the need for argon separation and recycling.

An embodiment is a method for converting hydrocarbon feed stream to hydrogen and solid carbon, optionally in an SMR unit/plant. The method comprises: delivering a feed stream comprising hydrocarbon into at least one plasma reactor; activating microwave pyrolysis in the plasma reactor to convert the hydrocarbon feed stream to an acetylene feed stream; delivering the acetylene feed stream to a refractory; and activating decomposition of the acetylene into hydrogen in the refractory. Acetylene decomposition may produce hydrogen, solid carbon, a mixture of polyaromatic molecules, and a tail gas. This decomposition may produce solid carbon, which, in turn, reduces CO₂ emissions. The term “solid carbon” as used herein includes carbon black, graphene sheets, nanocarbon materials containing graphene nanostructures as disclosed in International Patent Application Publication No. WO 2020/209975, which is incorporated by reference herein in its entirety, or any combination thereof. This decomposition may produce a mixture of polyaromatic molecules, which may include benzene, graphene, and other agglomerates of carbon that assemble into droplets, adhere and release hydrogen. The mixture of polyaromatic molecules may be a desired byproduct which may be used in the manufacture of other carbon materials, such as but not limited to, electrodes, synthetic graphite, and needles. Polyaromatic molecules are chemical compounds containing only carbon and hydrogen, and that include multiple aromatic rings.

Any known plasma reactor may be used in accordance with this disclosure. Any plasma reactor such as those disclosed in U.S. Pat. Nos. 9,095,835, 9,987,611, 10,363,542, 10,434,490, 11,021,661, US Patent Publication No. 2019/0047865, and US Patent Publication No. 2019/0046947, each of which is incorporated by reference herein in its entirety, may be used in accordance with this disclosure. The plasma reactor, which may also be referred to as a microwave plasma reactor, may eliminate flaring and reduce the carbon footprint of associated petroleum gas. The microwave plasma reactor technology addresses the need for a small footprint, modular, robust and energy efficient method for value-added utilization of waste or by-product gas streams, which are presently not valorized and instead are harmfully flared. These include industrial waste streams, such as those from coking plants or refineries, and methane/carbon dioxide-rich biogas produced by the landfills and manure management facilities of the agricultural industry. Microwave plasma enables a small-scale, low-cost process for direct, rapid and continuous conversion of hydrocarbon gases-ranging from pure methane to CO₂-rich effluent streams-into hydrogen and high-value solid carbons. Microwave plasma processes have lower energy requirements compared to conventional thermal plasmas, while offering reaction pathways not available for conventional chemical processes or thermal plasma reactors. The plasma reactor which performs microwave pyrolysis accepts a minimally processed gas stream to produce a single high value solid carbon product. The system is powered with electricity generated by a reciprocating engine genset, which utilizes the decarbonized effluent from the reactor and the balance of the waste gas.

Each plasma reactor may be powered with a single 100 kW microwave generator operating at 915 MHz frequency. At the target microwave energy requirement of 7 kWh-mw/kgCH₄, a single reactor would convert 14.3 kg of methane. Evaluation of conversion feasibility with carbon dioxide co-feeds (up to 50% on volumetric basis) have shown further reduction of the specific energy requirement (3 kWh-mw/kgFeed) as well as increase in the heating value of the output stream (up to 60%).

The conversion process with a plasma reactor provides a 37.61% net reduction in CO₂ emissions compared to flaring and enables non-negligible indirect reductions in CO₂ emissions, through the application of the solid carbon products in lightweighting additives (e.g., vehicles) and strengthening infrastructure.

The plasma reactor may be configured to receive a feed stream comprising hydrocarbons, such as, but not limited to, aromatic, alkylated aromatic, paraffinic, olefinic, cycloolefin, naphthenic, alkane, alkene, alkyl cycloalkane, alkylated cycloalkane, alkyne, alcohol, and heteroatom, and methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof and convert hydrocarbons in the feed stream to a product comprising a graphitic material in the presence of plasma. The plasma forming zone may include a radiation source, and a discharge tube coupled to the radiation source configured to receive a plasma forming material. The discharge tube may be made from a material that is transparent to the radio-frequency radiation. Optionally, the plasma forming material may include one or more of the following: argon, hydrogen, helium, neon, krypton, xenon, carbon dioxide, nitrogen, and water. A waveguide may be configured to couple the radiation source to the discharge tube. Alternatively, and/or additionally, the system may include a reaction tube configured to surround the discharge tube in the plasma forming zone to form an annulus. The feed stream flows in the annulus through the plasma forming zone. The feed stream may also comprise molecular hydrogen. Optionally, a molar ratio of the carbon containing species to the molecular hydrogen in the feed stream is about 5:1 to about 1:1. In various embodiments, the feed stream may include one or more of: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof. Additionally and/or alternatively, the feed stream may include: methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

In certain embodiments, the plasma forming material may include one or more first materials selected from the group consisting of: argon, hydrogen, helium, neon, krypton, xenon, carbon dioxide, nitrogen, and water.

The term “graphitic materials” refers to carbon containing solids including but not limited to: amorphous and graphitic carbon blacks of varying crystallinity, carbon onions and rosettes, graphite, graphene, functionalized graphene, and graphitic and graphenic carbon structures (containing one or more layers of graphene sheets), carbon nanotubes (CNTs), functionalized CNTs (or hybrid CNTs, denoted HNTs), and carbon fiber. The graphitic materials may be flat (completely flat and/or may include curved or curled sections), curved, curled, rosette shaped, spheroidal, or the like. In various embodiments, the graphitic material may be nano-graphene sheets, semi-graphitic particles, amorphous particles, or a combination thereof. The lateral dimensions of the nano-graphene sheets may be about 50 nm to about 500 nm. Additionally and/or alternatively, concentration of the nano-graphene sheets in the product may be proportional to a concentration of molecular hydrogen in the feed stream.

The plasma reactor may include a reaction zone configured to receive the plasma, receive feed stream comprising hydrocarbon, and convert the feed stream to a product comprising the graphitic materials in presence of the plasma. Plasma received in a reaction zone may initiate selective conversion of the feed stream to the product comprising graphitic materials. Products may also include hydrogen and/or chemicals such as ammonia. For example, the streamers or diffused the non-thermal plasma may act as an energy transfer catalyst activating the feed stream and enabling acceptance of additional microwave energy into the feed stream. The ions and electrons within the streamers or diffuse non-thermal plasma collide with the feed stream to selectively activate particular molecular modes resulting in an overall increase in energy efficiency compared with traditional thermodynamic or thermal-catalytic chemical dissociation. The collisions result in energy transfer sufficient to promote cleavage of a bond (e.g., hydrogen atom to a carbon atom bond) of the feed stream. For example, for the methane within the feed stream, the H₃C—H bond is cleaved by electron collisions.

In various embodiments, a feed stream comprising hydrocarbon may be first processed in a well-pad gas handling system. A typical well-pad gas handling system consists of flare headers, knockout drum, flashback seal, flare stack, and other onsite gas-handling infrastructure as required for maintenance and emergency procedures. For some well-pads, it may also include water and acid-gas scrubbers, natural gas liquid (NGL) knockout equipment, and other gas processing units.

The gas handling system evens out the flow rate, temperature and pressure. The gas converter's gas control manifold then distributes the feed stream between one or more microwave plasma reactors, where hydrocarbons are pyrolyzed into high-structure carbons as described above. The converted hydrogen-rich gas stream carries solid carbon particles into the carbon recovery system, comprised of cyclones and baghouses. After microwave pyrolysis and recovery of solid carbons, the effluent stream containing acetylene (referred to herein as an “acetylene feed stream” or “acetylene-containing feed stream”) is delivered to a refractory for further processing, hydrogen gas production, and reduction of CO₂ emissions.

Carbon materials are knocked out of the hot (1100 K-1350 K) effluent stream in a cyclone and are transported to a carbon processing facility. The cleaned stream enters a heat exchanger, where it pre-heats the natural gas feed, and is itself cooled prior to the final carbon knockout utilizing a baghouse collector.

Conventional pressure swing adsorption (PSA) will produce a high-purity hydrogen stream. The tail gas can be cycled back into the reactor or, if the microwave units augment a conventional SMR facility, supplied to the SMR furnace.

The microwave energy supplied to the plasma reactor rapidly (fraction of a second) may heat the supplied stream of feed stream comprising hydrocarbons (such as natural gas) to pyrolytic temperatures and a resulting acetylene feed stream including acetylene and optionally hydrogen and unconverted feed stream, at the temperatures >1100 K pass into the refractory. Any known means for connection and feeding of the acetylene feed stream, such as a nozzle injector, may be used to connect the plasma generator to the refractory. More than one plasma reactors may be coupled to the refractory, with each plasma reactor having a feed that connects the plasma reactor to the refractory for delivering an acetylene feed stream. The plasma reactors may be structured in any arrangement on top of or beside the refractory. In various embodiments, the plasma reactors may be arranged on top of a flat surface of the refractory in lines, or a in circular arrangement.

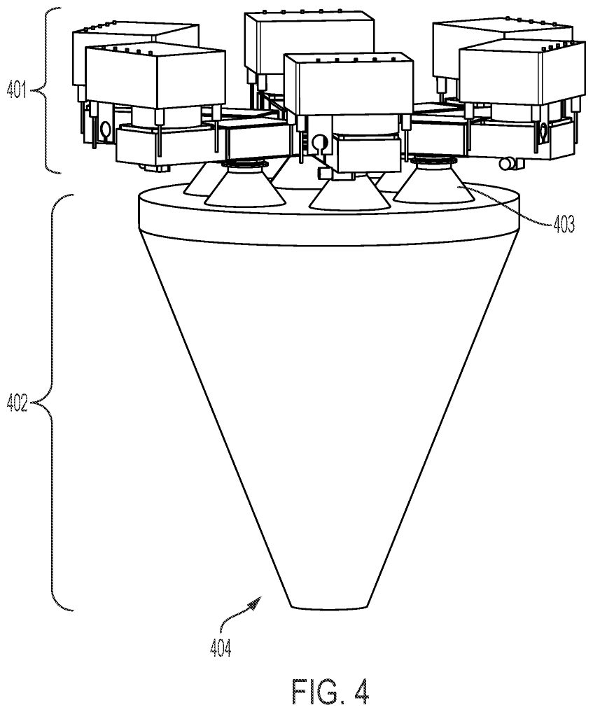

For examples, FIG. 4 depicts an example arrangement of plasma reactors and refractory. In FIG. 4, at least one plasma reactor 401 is coupled to the refractory 402. One to thirty (or more) plasma reactors 401 may be coupled to the refractory 402. Each plasma reactor 401 is powered by one or more microwave generators. The refractory 402 is configured to decompose acetylene to hydrogen and solid carbon. The refractory 402 may include one inlet 403 coupled to each plasma reactor 401 for receiving the acetylene feed stream. The inlet 403 may be at one end or at the top of the refractory, and at least one outlet 404 may be on a side or bottom of the refractory for expelling the hydrogen. In various embodiments, the refractory may comprise one or more auxiliary inlet gas feeds configured to feed a second feed stream comprising methane to the refractory.

In the refractory, acetylene proceeds to polycondense into carbon structures in the gas phase, producing molecular hydrogen and releasing energy (e.g., about 226.88 kJ/mol) back into the gas stream. This energy is available to drive further hydrocarbon pyrolysis. For instance, on the basis of enthalpies alone, for each decomposed acetylene molecule, 3 methane molecules may undergo complete decomposition to solid carbon and hydrogen.

There may be two types of feeds delivered into the refractory: i) one acetylene feed stream from each plasma reactor; and ii) an auxiliary feed stream comprising delivered directly to the refractory without passing through the plasma reactor. In various embodiments, the auxiliary or “second” feed stream may include one or more hydrocarbon from the following: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof. Additionally and/or alternatively, the feed stream may include: methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

In various embodiments, the acetylene feed stream may contain about 10 vol. % to about 70 vol. %, about 15 vol. % to about 60 vol. %, or about 20 vol. % to about 50 vol. %, acetylene. The acetylene feed stream may also include one or more other gases selected from hydrogen, ethane, methane, ethylene, and any combination thereof. In some embodiments, the acetylene feed stream may be delivered into the refractory at a flow rate at about 30 to about 250 standard liters per minute (slpm), about 30 to about 50 slpm, about 50 to about 100 slpm, about 100 to about 150 slpm, about 150 to about 200 slpm, or about 200 to about 250 slpm.

In various embodiments, the auxiliary feed stream may comprise methane gas that provide 100 vol. % methane gas into the refractory so that the molar ratio of methane to acetylene (CH₄:C₂H₂) inside the refractory is about 3:1. In various embodiments, the methane feed stream may act as both a physical quench and a chemical quench; to limit the reaction and cools the temperature in the refractory. In certain embodiments, there are more than one inlet feeds for the auxiliary feed stream, which may be placed along the sides of the refractory and create a swirl of gases inside the reactor upon entry. One or more inlet feeds for the auxiliary feed stream may be placed on the top of the refractory, or there may be one or more inlet feed on a side and another on the top of the refractory.

Once the gases are mixed within the refractory, there may be an acetylene concentration of about 10 vol. % to about 30 vol. %, about 15 vol. % to about 25 vol. %. or about 18 vol. % to about 22 vol. %.

There may be one or two outputs on the refractory to remove products after activation and processing therein: i) solid carbon, a mixture of polyaromatic molecules, or a combination thereof from hydrocarbon decomposition; and ii) a gas stream of hydrogen (e.g., produced from hydrocarbon decomposition), unconverted feed stream such as unconverted methane, acetylene, and other co-products. The production of solid carbon and/or polyaromatics is a means to capture the carbon from the initial feed stream and provide a form that has a commercial value, no sequestration costs and provides a by-product credit for hydrogen production.

The output gas stream may contain about 90% H₂ by volume, with the balance of gases comprised primarily of unconverted feed stream such as unconverted methane, acetylene, and also ethylene, ethane, and higher hydrocarbons.

The pressure in the refractory may be about 12 PSI to about 73 PSI, or about 12 PSI. Higher pressures make it easier for acetylene to condense speeding up the process and reducing the energy requirement. The pressure in the refractory may be up to about 5 atmospheres.

The temperature within the refractory may be about 800° C. to about 1600° C., about 1000° C. to about 1300° C., about 1000° C., or about 1200° C. In various embodiments, the energy required to decompose the auxiliary feed stream may be provided by the energy released from the exothermic decomposition of acetylene stream into carbon and hydrogen.

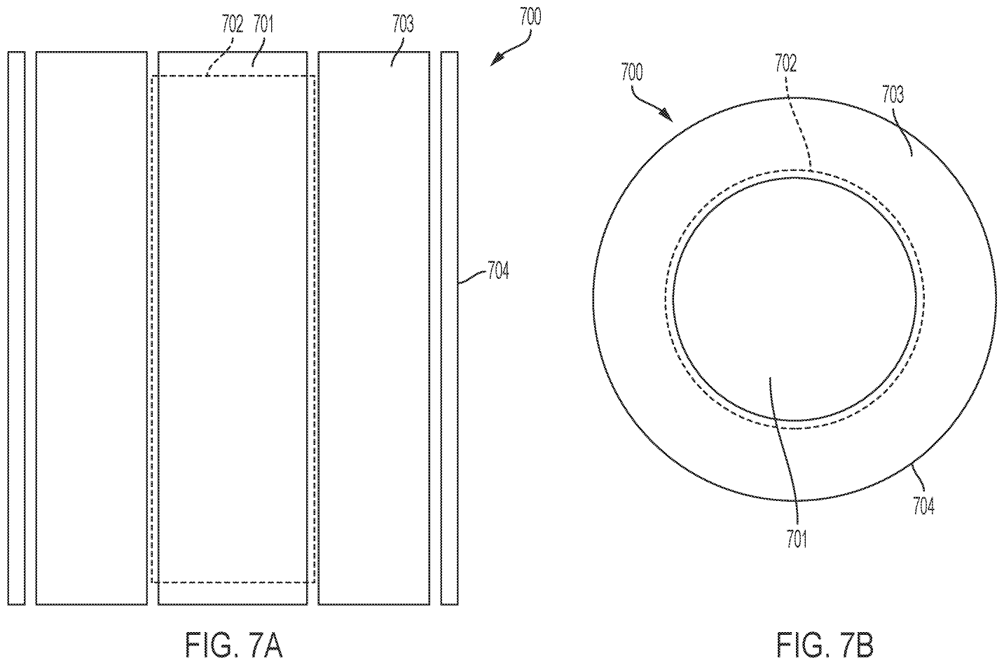

As shown in the top and side views of FIGS. 7A and 7B, in some embodiments, the refractory may include a refractory subsystem 700 comprising an alumina tube 701 configured to receive the output gas stream, e.g., acetylene stream from the plasma reactor and release products after activation and processing of the acetylene stream. In various embodiments, the alumina tube 701 may be shaped of various geometries including a cylindrical shape, a tapering conical shape, a rectangular shape, a square shape, or convergent-divergent cone shape. In other embodiments, the refractory may also include an additional heating element 702 or elements such as conduction heater, heating belt, infrared lamp, nichrome wire, or nichrome coil coupled to the outer surface of the alumina tube 701 to provide additional energy to drive the decomposition reaction in the refractory. In various embodiments, the external heating elements 702 are configured to heat the outer refractory wall to about 700° C. to about 1000° C., about 750° C. to about 850° C., about 850° C. to about 950° C., or about 950° C. to about 1000° C.

In certain embodiments, the outer surface of the alumina tube 701 maybe enclosed by one or more insulating layer 703 to prevent the heat from the alumina tube to escape through the outer metallic walls of the refractory. In various embodiments, the insulating layer 703 may comprise any thermally resistant material such as ceramic wool/fiber such as Kaowool that is capable of insulating up to about 1500° C. In various embodiments, the insulating layer 703 may be about 1 to about 5 inches thick, or about 2 to about 4 inches thick, or about 3 inches thick. In various embodiments, an outer housing 704 configured to match the dimension and the geometry of the alumina tube 701, external heating unit 702, and thermally insulating layer 703 may be used to house the internal components of the refractory. In some embodiments, outer housing 704 may comprise metals such as but not limited to stainless steel, aluminum, aluminum alloy, copper, copper alloy, bronze, tungsten, titanium, and alloys of iron such as steel-high carbon, steel-medium carbon, or steel-low carbon. In other embodiments, any metal or material that is structurally and thermally stable at temperatures above about 200° C. may be utilized. In some embodiments, the wall thickness of outer housing 704 may be about 0.065 inches to about 0.134 inches. In some embodiments, the refractory may include an insulated connection, or a “neck” between the plasma reactor and the heated refractory to provide additional thermal insulation and structural stability at the plasma reactor-refractory junction. In various embodiments, the neck may comprise thermally insulating materials including for example, alumina casting fortified with mullite powder to enhance the thermal and structural properties.

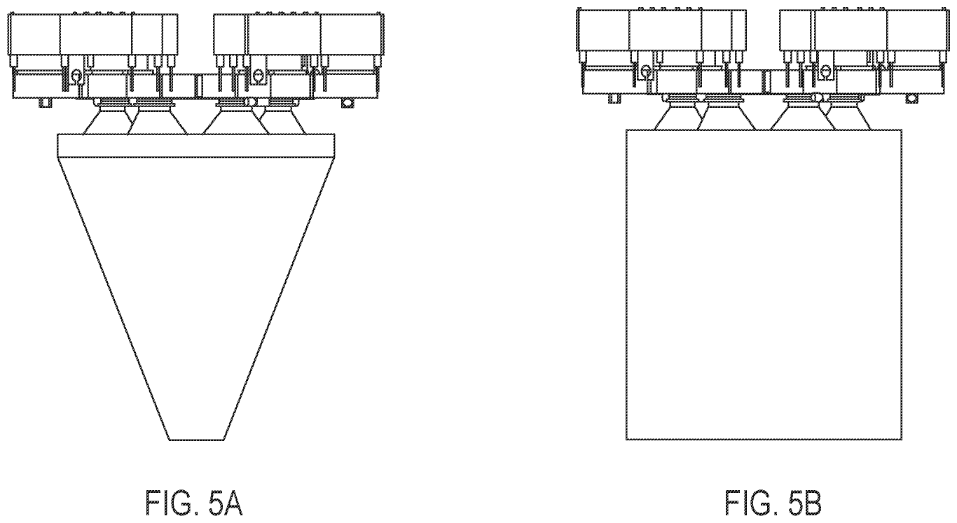

In various embodiments, the refractory may be assembled of any size and shape to achieve the desired residence time. The shape of the refractory may be a cylindrical shape (as shown in FIG. 5A), a conical shape (as shown in FIG. 5B), a rectangular shape, a square shape, a convergent-divergent cone shape, or combination thereof. The refractory may be a 100 kW (kilowatt) to a 4000 kW system. The total power of the reactors may provide a 100 kW, 200 kW, 300 kW, 400 kW, 500 kW, 600 kW, 700 kW, or 800 kW system. The refractory may be a 1.0 MW (megawatt), 1.5 MW, 1.8 MW, 2.0 MW, 2.4 MW, 2.6 MW, or 3.0 MW system. The refractory may be a 600 kW conical system, about 1.5 to about 3 meters, or about 2 meters in height. The volume of the refractory may be about 1.0 m³ to about 5.5 m³, about 1.0 m³ to about 4.5 m³, or about 1.0 m³ to about 4.0 m³.

The acetylene decomposition described herein may be carried out in the presence or absence of a catalyst. In various embodiments, a catalyst may be added to the refractory to promote or facilitate decomposition of acetylene. The catalyst material may include a high surface carbon, such as a graphitic material or carbon black. In various embodiments, when a feed stream containing acetylene and hydrogen is passed into the refractory containing a catalyst (e.g., activated carbon), a higher percentage of acetylene decomposes in a shorter amount of time.

In various embodiments, acetylene decomposes and produces solid carbon which is hot and acts as an energy transfer agent and catalyst for further decomposition of acetylene and hydrocarbons. The solid carbon may be carbon black, graphene sheets, nanocarbon materials as disclosed in International Patent Application Publication No. WO 20/209975, or a combination thereof. Carbon is radiative and transmits heat to methane. This carbon catalyst allows hydrocarbons to decompose at a lower temperature than it would in the absence of carbon catalyst. As such, it may be a self-catalyzing process; carbon is produced and catalyzes. Optionally, solid carbon may be injected into the refractory during processing to act as a catalyst.

The catalyst may lower the temperature in the refractory by more than about 200 degrees or more than about 300° C. The catalyst may lower the temperature in the refractory by about 200° C. to about 500° C., or about 300° C. to about 400° C.

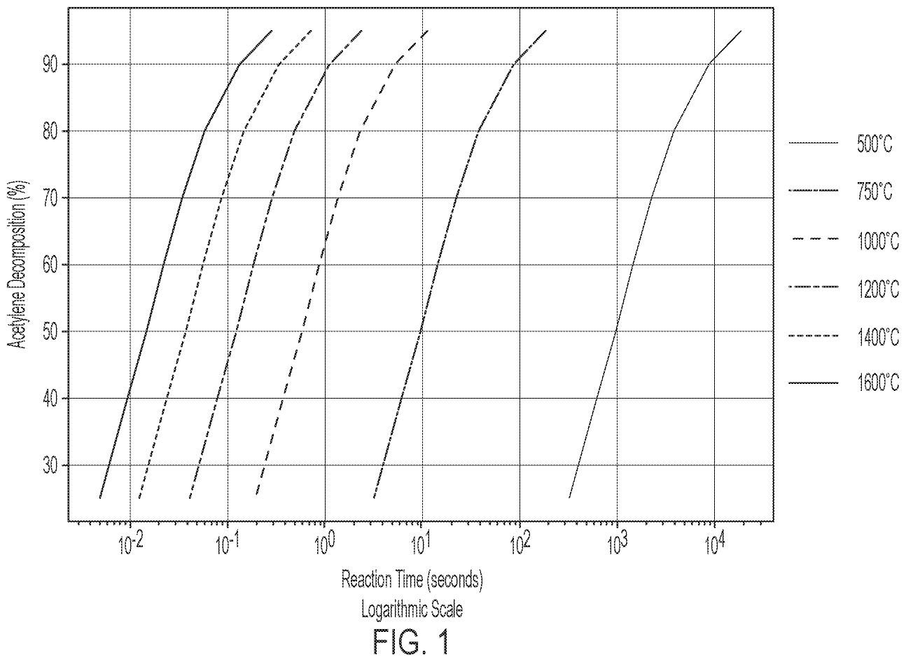

FIG. 1 shows example acetylene decomposition as a function of temperature and time in a refractory. Residence time is the amount of time in the refractory at a set temperature to promote acetylene decomposition. Accordingly, residence time in a refractory also may be referred to as a reaction time. In some embodiments, residence time may be about 0.1 to about 30 seconds. The residence time may be about 0.5 seconds to about 10 seconds, or about 3 seconds to about 6 seconds. In certain embodiments, about 50 vol. % to about 100 vol. %, about 60 vol. % to about 100 vol. %, about 70 vol. % to about 100 vol. %, about 70 vol. % to about 95 vol. %, about 75 vol. % to about 90 vol. %, or about 80 vol. % of the acetylene may decompose in under about 30 seconds.

Acetylene decomposition is a rapid second order chain polymerization reaction even when acetylene is highly diluted. At about 1000° C., there is about 80% acetylene decomposition after about 1.5 seconds to about 3 seconds, or about 2.37 seconds for about 80% acetylene decomposition, and there is about 98% acetylene decomposition after about 25 seconds to about 30 seconds, or about 29 seconds.

At about 1200° C., there is about 80% acetylene decomposition after about 0.3 second to about 1.5 seconds, or about 0.5 seconds, and there is about 98% acetylene decomposition after about 5 second to about 10 seconds, or about 6 seconds.

For example, at 0.5 vol. % acetylene concentration in a gas stream, acetylene decomposition at about 1200° C. is fast and accelerates with residence time: at 1 msec: about 1% converted; at 56 msec: about 7% converted; and at 112 msec: about 23% converted.

At 10 vol % acetylene and 77 vol % hydrogen in a gas stream, at about 1000 K (700° C.), about 50% acetylene decomposes in about 4 seconds.

At 10 vol % acetylene and 77 vol % hydrogen in a gas stream, at about 1500° C., about 100% acetylene decomposes in about 4 seconds.

In various embodiments, methane decomposition may be supported with the heat of reaction from acetylene decomposition. The energy (heat) from the decomposition of 1 mole of acetylene may decompose 3 moles of methane. For methane decomposition, at about 900° C., there is no decomposition without a catalyst. At about 1200° C., there is rapid decomposition even diluted without a catalyst: greater than about 80% conversion in less than about 2 seconds. At about 1200° C., there is about 80% methane decomposition after about 0.5 second to about 2 seconds, or about 1 second, and there is about 85% methane decomposition after about 1 second to about 2 seconds, or about 1.5 seconds.

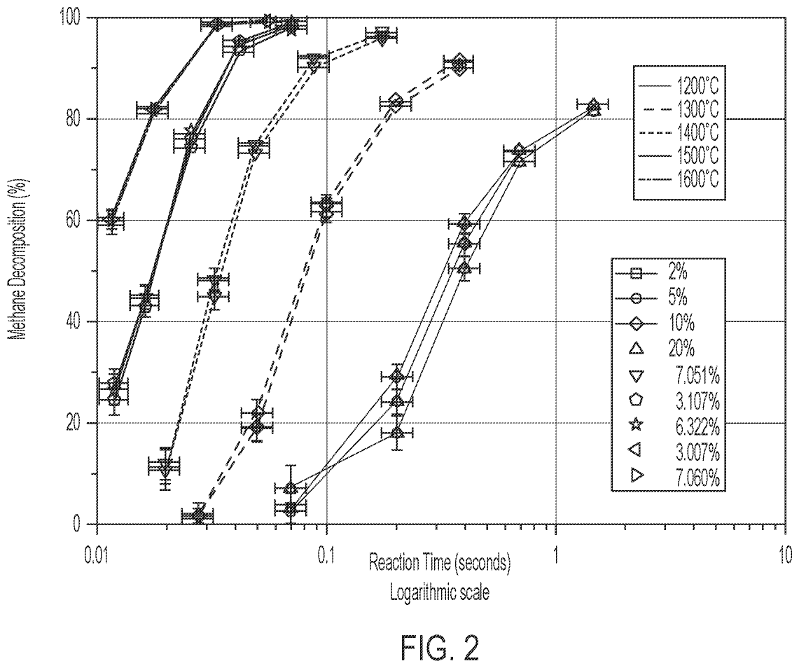

FIG. 2 shows example methane decomposition as a function of temperature and time in a refractory.

Similarly, FIG. 2 shows example methane decomposition as a function of temperature and time in a refractory. In some embodiments, residence time may be about 0.05 to about 2.5 seconds. The residence time may be about 0.05 seconds to about 2 seconds, 0.25 seconds to about 1.5 seconds, about 0.5 seconds to about 1 seconds, or about 0.75 seconds. In certain embodiments, about 50 vol. % to about 100 vol. %, about 60 vol. % to about 100 vol. %, about 70 vol. % to about 100 vol. %, about 70 vol. % to about 95 vol. %, about 75 vol. % to about 90 vol. %, or about 80 vol. % of the methane may decompose in under about 3 seconds.

At about 1200° C., there is about 80% methane decomposition after about 1 second to about 1.5 seconds. At about 1300° C., about 90% methane decomposes in about 0.5 seconds. At about 1400° C., about 95% methane decomposes in about 0.3 seconds. At about 1500° C., about 100% methane decomposes in about 0.05 seconds and at about 1600° C., about 100% methane decomposes in about 0.05 seconds.

Another embodiment is a system for producing hydrogen from a feed stream comprising hydrocarbon, with reduced CO₂ emissions. The system includes: at least one plasma reactor; and a refractory coupled to the at least one plasma reactor.

The terms used in connection with this embodiment have the same meanings and definitions as discussed above.

The features and advantages of the present disclosure are more fully shown by the following examples which are provided for purposes of illustration and are not to be construed as limiting the invention in any way.

### EXAMPLES

### Example 1

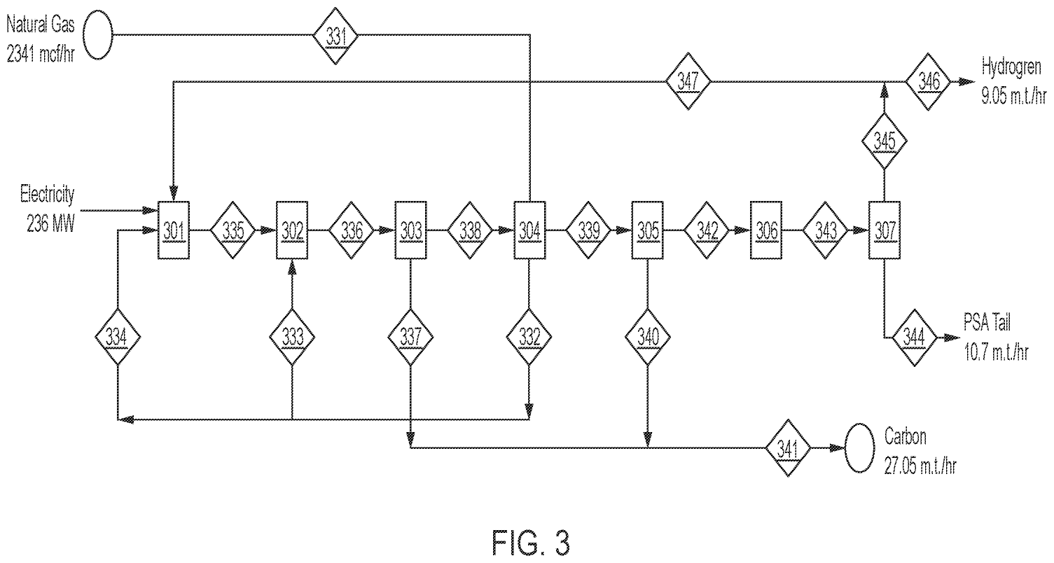

In an example system, as shown in FIG. 3, a microwave pyrolysis reactor (301) is powered by one or more 100 kW microwave generators 350, with 75% assumed electric efficiency (82% nominal vendor-stated efficiency), each consuming 133 kW of electric power.

Plasma Pyrolysis of methane to acetylene

2CH₄→3H₂+C₂H₂ ΔH=376.08 kJ/mol C₂H₂

The supplied stream of natural gas (334) is rapidly converted in reactor 301 to C2s and hydrogen (335) and at bulk temperatures >800° C. pass into the refractory (302). In the refractory, acetylene proceeds to polycondense into carbon structures in the gas phase, producing molecular hydrogen and outputting 226.88 kJ/mol of energy back into the gas stream. This energy is available to drive additional pyrolysis of auxiliary methane (333) stream.

Acetylene and Methane Decomposition

2CH₄→3H₂+C₂H₂ ΔH=376.08 kJ/mol C₂H₂

C₂H₂→H₂+2C(s) ΔH=−226.88 kJ/mol C₂H₂

Solid carbon entrained in the pyrolysis product stream (336) is mostly removed in the cyclone (303) and is transported (337, 341) to carbon processing. The cleaned stream (8) is cooled in the heat exchanger (304) by the incoming natural gas (331) and passes into cooling and baghouse assembly (305), where the gas is cooled to ambient temperature and remaining carbon particles are removed and transported to carbon processing (340, 341).

Finally, the 90% hydrogen stream (342) is pressurized to 20 bar (306) and purified to 99.997% (345) with pressure-swing adsorption (PSA) (307). A small (80 kg) amount is cycled back (347) for use in microwave pyrolysis reactor (301). Recycling C2-rich PSA tail gas into the pyrolysis reactors can reduce both specific energy requirement and natural gas consumption.

TABLE 1a
Feed Streams in an Example Process Flow according to FIG. 3

| Streams | 1 | 2 | 3 | 4 | 5 | 6 | 7 | 8 | 9 |
|---|---|---|---|---|---|---|---|---|---|
| Temperature (C.) | 25 | 700 | 700 | 700 | 1000 | 1000 | 1000 | 1000 | 300 |
| Pressure (bar) | 3.5 | 3.5 | 3.5 | 1.0 | 1.0 | 1.0 | 1.0 | 1.0 | 1.0 |
| Molar Flow (kmol/hr) | 2919 | 2919 | 953 | 1966 | 3327 | 7767 | 2230 | 5537 | 5537 |
| Mass Flow (m.t./hr) | 46.85 | 46.85 | 15.30 | 31.55 | 31.63 | 46.93 | 26.78 | 20.15 | 20.15 |
| Methane | 100% | 100% | 100% | 100% | 29.9% | 10.6% | 0.0% | 24.6% | 24.6% |
| Hydrogen | 0.0% | 0.0% | 0.0% | 0.0% | 13.1% | 21.6% | 0.0% | 50.4% | 50.4% |
| Ethylene | 0.0% | 0.0% | 0.0% | 0.0% | 4.3% | 2.9% | 0.0% | 6.7% | 6.7% |
| Ethane | 0.0% | 0.0% | 0.0% | 0.0% | 0.3% | 0.2% | 0.0% | 0.5% | 0.5% |
| Acetylene | 0.0% | 0.0% | 0.0% | 0.0% | 52.4% | 7.1% | 0.0% | 16.4% | 16.4% |
| Solid Carbon | 0.0% | 0.0% | 0.0% | 0.0% | 0.0% | 57.6% | 100% | 1.3% | 1.3% |
| Total | 100% | 100% | 100% | 100% | 100% | 100% | 100% | 100% | 100% |

TABLE 1b
Feed Streams in an Example Process Flow according to FIG. 3 cont.

| Streams | 10 | 11 | 12 | 13 | 14 | 15 | 16 | 17 |
|---|---|---|---|---|---|---|---|---|
| Temperature (C.) | 35 | 35 | 35 | 35 | 35 | 35 | 35 | 35 |
| Pressure (bar) | 1.0 | 1.0 | 1.0 | 20.0 | 1.0 | 20.0 | 20.0 | 20.0 |
| Molar Flow (kmol/hr) | 23 | 2252 | 5515 | 5515 | 990 | 4525 | 4484 | 41 |
| Mass Flow (m.t./hr) | 0.27 | 27.05 | 19.88 | 19.88 | 10.74 | 9.14 | 9.06 | 0.08 |
| Methane | 0.0% | 0.0% | 24.9% | 24.9% | 46.1% | 0.0% | 0.0% | 0.0% |
| Hydrogen | 0.0% | 0.0% | 51.1% | 51.1% | 9.5% | 100.0% | 100.0% | 100.0% |
| Ethylene | 0.0% | 0.0% | 6.8% | 6.8% | 12.6% | 0.0% | 0.0% | 0.0% |
| Ethane | 0.0% | 0.0% | 0.5% | 0.5% | 1.0% | 0.0% | 0.0% | 0.0% |
| Acetylene | 0.0% | 0.0% | 16.7% | 16.7% | 30.9% | 0.0% | 0.0% | 0.0% |
| Solid Carbon | 100% | 100% | 0.0% | 0.0% | 0.0% | 0.0% | 0.0% | 0.0% |
| Total | 100% | 100% | 100% | 100% | 100% | 100% | 100% | 100% |

As shown in Tables 1a and 1b, stream 5 entering the refractory (302) contains 29.9% methane, 13.1% hydrogen, 52.4% acetylene, and 0% solid carbon. In addition, an auxiliary feed stream of pure methane 333 (100% methane) is shown entering the refractory 302 without being rerouted through microwave pyrolysis reactor 301. After processing in the refractory, the output stream 6 contains 10.6% methane, 21.6% hydrogen, 7.1% acetylene, and 57.6% solid carbon. Accordingly, by comparing the amount of each of the components in streams 5 and 6, processing in the refractory has converted methane and acetylene to hydrogen and solid carbon.

### Example 2

Conversion of CH₄, C₂H₆, C₃H₈, CO₂, and mixtures into hydrocarbon and solid carbon products.

TABLE 2
Conversion of various hydrocarbon feed stream according to Feed Streams

| | | Conversion | Energy | Product | Product | Product | Product |
|---|---|---|---|---|---|---|---|
| Starting Gas Feed | Flow Rate | efficiency | requirement* | C₂H₂ | Carbon | C₂H₄ | C₂H₆ |
| CH₄ only | 5-120 slpm | 10-94% | 7.2-20 | 10-70% | 5-92% | 2-10% | 1-5% |
| CH₄ only | 30 slpm | 42% | 10.2 | 58% | 35% | 6% | 1% |
| C₂H₆ only | 25 slpm | 40% | 7.5 | 40% | 22% | 30% | 8% |
| C₃H₈ only | 25 slpm | 35% | 5.8 | 30% | 35% | 20% | 15% |
| CH₄:C₂H₆ (0-50%) | 30 slpm | 20-25% | 7-12 | 40-62% | 35-40% | 5-20% | 1-5% |
| CH₄:C₃H₈ (0-100%) | 30 slpm | 15-35% | 6-14 | 35-65% | 15-38% | 0-22% | 1-10% |
| Mix 1: CH₄:C₂H₆:C₃H₈:CO₂ | 25-30 slpm | 30-40% | 9.5-12 | 50-55% | 20-25% | 20-25% | 1-3% |
| (73.9%:13.3%:7.8%:5.0%) | | | | | | | |
| Mix 2: CH₄:C₂H₆:C₃H₈:CO₂ | 35 slpm | 30-35% | 7.5-8 | 40-42% | 35-40% | 20% | 1-5% |
| (48.1%:18.8%:29.1%:4.0%) | | | | | | | |

*kWh/kg-converted feed gas

Additional experiments utilizing various hydrocarbons and their mixtures as the starting feed stream for conversion into hydrocarbon and solid carbon products were performed. As shown in Table 2, introducing pure methane (CH₄) stream at a flow rate at about 5 to 120 slpm resulted in a product conversion of about 10 to 94% of methane into 10 to 70% C₂H₂, 5 to 92% solid carbon, 2 to 10% C₂H₄, and 1 to 5% of C₂H₆. Standardizing the methane flow to about 30 slpm resulted in a product conversion of 42% yielding 58% C₂H₂, 35% solid cardon, 6% C2H₄, and 1% C₂H.

Utilizing ethane (C₂H₆) resulted in 40% conversion of the starting feed yielding 40% C₂H₂, 22% solid cardon, 30% C₂H₄, and 8% C₂H₆. Under identical flow rate of 25 slpm, longer length hydrocarbons, e.g., propane (C₃H₈) resulted in greater product selectivity towards the C₂ species (ethylene and ethane). C₃H₈ under flow rate of 25 slpm resulted in 35% conversion into product yielding 30% C₂H₂, 35% solid cardon, 20% C₂H₄, and 15% C₂H.

Binary mixtures of CH₄:C₂H₆ and CH₄:C₃H₈ were independently introduced to the microwave pyrolysis reactor, then delivered to the refractory for decomposition into hydrocarbon and solid carbon products. The CH₄:C₂H₆ mixture resulted in product conversion of 20-25% yielding 40-62% C₂H₂, 35-40% solid carbon, 5-20% C₂H₄, and 1-5% C₂H₆ while the CH₄:C₃H₈ mixture resulted in 15-35% product conversion into 35-65% C₂H₂, 15-38% solid carbon, 0-22% C₂H₄, and 1-10% C₂H.

Two different mixtures of CH₄, C₂H₆, C₃H₈, and CO₂, comprising different volume ratios were decomposed to determine the product selectivity of the decomposed feed mixtures. Mixture 1, comprising 73.9% CH₄, 13.3% C₂H₆, 7.8% C₃H₈, and 5.0% CO₂, resulted in 30-40% product conversion yielding 50-55% C₂H₂, 20-25% solid cardon, 20-25% C₂H₄, and 1-2% C₂H₆ while Mixture 2, comprising 48.1% CH₄, 18.8% C₂H₆, 29.1% C₃H₈, and 4.0% CO₂, resulted in 30-35% product conversion yielding 40-42% C₂H₂, 35-40% solid cardon, 20% C₂H₄, and 1-5% C₂H. Accordingly, by varying the amount of starting hydrocarbon feed stream and flow rate, the product selectivity can be tuned to achieve desired hydrocarbon or solid carbon products and the quantity.

It should be understood that the foregoing description is only illustrative of the present disclosure. Various alternatives and modifications can be devised by those skilled in the art without departing from the present disclosure. Accordingly, the present disclosure is intended to embrace all such alternatives, modifications and variances which fall within the scope of the appended claims.

The features from different embodiments disclosed in this document may be freely combined. For example, one or more features from a method embodiment may be combined with any of the product embodiments. Similarly, features from a product embodiment may be combined with any of the method embodiments disclosed in this document.

Without excluding further possible embodiments, certain example embodiments are summarized in the following clauses:

Clause 1: A method for producing hydrogen, the method comprising: delivering a first feed stream into a plasma reactor; activating microwave pyrolysis in the plasma reactor to convert the first feed stream to an acetylene-containing feed stream; delivering the acetylene-containing feed stream to a refractory; and activating decomposition of the acetylene-containing feed stream into hydrogen in the refractory.

Clause 2: The method of clause 1, wherein the first feed stream comprises one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof.

Clause 3: The method of clause 1 or clause 2, wherein the first feed stream further comprises methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

Clause 4: The method of any of the preceding clauses, further comprising delivering a second feed stream to the refractory.

Clause 5: The method of clause 4, wherein the second feed stream comprises one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof.

Clause 6: The method of clause 4 or clause 5, wherein the second feed stream further comprises methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

Clause 7: The method of any of the preceding clauses, wherein a temperature within the refractory is at about 800° C. to about 1600° C.

Clause 8: The method of clause 7, wherein the temperature within the refractory is at about 1000° C. to about 1300° C.

Clause 9: The method any of the preceding clauses, wherein a catalyst is present in the refractory.

Clause 10: The method of clause 9, wherein the catalyst is solid carbon.

Clause 11: The method of clause 1, wherein the decomposition of the acetylene-containing feed stream produces the hydrogen and solid carbon.

Clause 12: The method of any of clauses 4-6, wherein decomposition of the second feed stream produces hydrogen and solid carbon.

Clause 13: A system for producing hydrogen from a feed stream comprising hydrocarbons, the system comprising: a plasma reactor for converting the feed stream to an acetylene-containing feed stream; and a refractory coupled to the plasma reactor.

Clause 14: The system of clause 13, wherein the feed stream comprises one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof.

Clause 15: The system of clause 13 or clause 14, wherein the feed stream further comprises methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

Clause 16: The system of any of clauses 13-15, comprising one or more additional plasma reactors coupled to the refractory.

Clause 17: The system of any of clauses 13-16, wherein the refractory comprises at least one inlet coupled to the plasma reactor for receiving the acetylene-containing feed stream, the inlet being at one end of the refractory, and at least one outlet at an opposite end of the refractory for expelling the hydrogen.

Clause 18: The system of any of clauses 13-17, wherein the refractory is configured to decompose the acetylene-containing feed stream to the hydrogen and solid carbon.

Clause 19: The system of any of clauses 13-18, wherein the refractory further comprises one or more auxiliary inlet gas feeds configured to feed a second feed stream.

Clause 20: The system of clause 19, wherein the second feed stream comprises one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof.

Clause 21: The system of clause 19 or clause 20, where the second feed stream further comprises methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

Clause 22: The system of any of clauses 19-21, where the refractory is configured to decompose the second feed stream to hydrogen and solid carbon.

Clause 23: The system of any of clauses 13-22, further comprising a heating element attached to an outer surface of the refractory.

Clause 24: The system of any of clauses 13-23, further comprising an insulating layer enclosing an outer surface of the refractory.

Clause 25: The system of any of clauses 13-24, wherein the refractory is a conical shape, a rectangular shape, a square shape, a cylindrical shape, a converging-diverging cone shape, or a combination thereof.

What is claimed is:

1. A method for producing hydrogen, the method comprising: delivering a first feed stream into a plasma reactor;
activating microwave pyrolysis in the plasma reactor to convert the first feed stream to an acetylene-containing feed stream;
delivering the acetylene-containing feed stream to a refractory; and
activating decomposition of the acetylene-containing feed stream into hydrogen in the refractory.

2. The method of claim 1, wherein the first feed stream comprises one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof.

3. The method of claim 1, wherein the first feed stream further comprises methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

4. The method of claim 1, further comprising delivering a second feed stream to the refractory.

5. The method of claim 4, wherein the second feed stream comprises one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof.

6. The method of claim 4, wherein the second feed stream further comprises methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

7. The method of claim 1, wherein a temperature within the refractory is at about 800° C. to about 1600° C.

8. The method of claim 7, wherein the temperature within the refractory is at about 1000° C. to about 1300° C.

9. The method of claim 1, wherein a catalyst is present in the refractory.

10. The method of claim 9, wherein the catalyst is solid carbon.

11. The method of claim 1, wherein the decomposition of the acetylene-containing feed stream produces the hydrogen and solid carbon.

12. The method of claim 4, wherein decomposition of the second feed stream produces hydrogen and solid carbon.

13. A system for producing hydrogen from a feed stream comprising hydrocarbons, the system comprising:
a plasma reactor for converting the feed stream to an acetylene-containing feed stream; and
a refractory coupled to the plasma reactor.

14. The system of claim 13, wherein the feed stream comprises one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof.

15. The system of claim 13, wherein the feed stream further comprises methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

16. The system of claim 13, comprising one or more additional plasma reactors coupled to the refractory.

17. The system of claim 13, wherein the refractory comprises at least one inlet coupled to the plasma reactor for receiving the acetylene-containing feed stream, the inlet being at one end of the refractory, and at least one outlet at an opposite end of the refractory for expelling the hydrogen.

18. The system of claim 13, wherein the refractory is configured to decompose the acetylene-containing feed stream to the hydrogen and solid carbon.

19. The system of claim 13, wherein the refractory further comprises one or more auxiliary inlet gas feeds configured to feed a second feed stream.

20. The system of claim 19, wherein the second feed stream comprises one or more of the following hydrocarbons: aromatic, alkylated aromatic, paraffinic, olefinic, cycloolefin, naphthenic, alkane, alkene, alkyl cycloalkane, alkylated cycloalkane, alkyne, alcohol, and heteroatom, and a combination thereof.

21. The system of claim 19, where the second feed stream further comprises methane, ethane, propane, butane, syngas, natural gas, biogas, flue gas, methanol, ethanol, propanol, butanol, hexane, benzene, paraffin, naphthalene, styrene, vinyl chloride, 1,2-dichloroethane, allyl alcohol, propionaldehyde, vinyl bromide, or a combination thereof.

22. The system of claim 19, where the refractory is configured to decompose the second feed stream to hydrogen and solid carbon.

23. The system of claim 13, further comprising a heating element attached to an outer surface of the refractory.

24. The system of claim 13, further comprising an insulating layer enclosing an outer surface of the refractory.

25. The system of claim 13, wherein the refractory is a conical shape, a rectangular shape, a square shape, a cylindrical shape, a converging-diverging cone shape, or a combination thereof.
